(19) United States
(12) Patent Application Publication
(10) Pub. No.: US 2016/0025711 A1
(43) Pub. Date: Jan. 28, 2016

(54) FLUORESCENT MARKERS AND METHODS FOR IMAGING DISEASES

(71) Applicants: Robin L. MCCARLEY, (US); William C. SILVERS, (US)

(72) Inventors: Robin L. McCarley, Prairieville, LA (US); William C. Silvers, Baton Rouge, LA (US)

(73) Assignee: BOARD OF SUPERVISORS OF LOUISIANA STATE UNIVERSITY AND AGRICULTURAL AND MECHANICAL COLLEGE, Baton Rouge, LA (US)

(21) Appl. No.: 14/775,967

(22) PCT Filed: Mar. 25, 2014
(86) PCT No.: PCT/US14/31657

§ 371 Date: Sep. 14, 2015

Related U.S. Application Data
(60) Provisional application No. 61/804,961, filed on Mar. 25, 2013.

(51) Int. Cl. G01N 33/52; A61K 49/00; C07D 221/14

(52) U.S. Cl. CPC G01N 33/52; C07D 221/14; A61K 49/0021; A61K 49/0052

(57) ABSTRACT

Methods and fluorescent probes that detect the presence of diseased cells such as cancer. The fluorescent probes are cloaked, turn-on probes having the fluorescence reporter released only in the presence of enzymes typically over-expressed in diseased and cancerous tissue. Probes include a fluorescent napthalimide reporter with a quinoidal moiety cloak.

### CROSS-REFERENCE TO RELATED APPLICATIONS

This application claims benefit of U.S. Provisional Application 61/804,961 filed Mar. 25, 2013, which is incorporated herein by reference.

### STATEMENT REGARDING FEDERALLY SPONSORED RESEARCH OR DEVELOPMENT

The invention was made with U.S. Government support under grant R21 CA135585 awarded by the National Institutes of Health and grant CHE 0910845 awarded by the National Science Foundation. The U.S. Government has certain rights in the invention.

### FIELD

The present invention relates to fluorescent markers and methods for imaging diseases, and in particular though non-limiting embodiments, to cloaked fluorophores which activate in the presence of enzymes present in tumor tissues and inflammatory diseases.

### BACKGROUND

Molecular probes whose fluorescent reporter signal is generated by enzyme activation (turn-on probes) hold great potential for identification, enumeration and study of living cancer cells and/or diseased tissues. Such probes may be invaluable for accurate and early diagnoses and optimization of surgical and personalized chemotherapeutic treatments. In particular, successful development of enzyme-activatable probes that can yield rapid, highly sensitive, and selective reporting of species or events associated with cancer cells may allow for definition of diseased and healthy tissue borders during fluorescence-assisted surgical resection of cancerous tissues. Enzyme-activatable probes may also provide and/or enhance collection of real-time information on tumor cell microenvironment or the pharmacodynamic effect of drugs on specific tumor cells.

To date, live cancer cell detection with varying degrees of selectivity and sensitivity has generally been limited to routes employing extracellular or cell-surface protein recognition of a covalently attached component of the probe or reporter. However, living cancer cells may also be identified and their type differentiated by targeting endogenous, intracellular, cancer-associated enzymes. Hydrophobic small-molecule (<1000 Da), turn-on probes provide a possible vehicle for such identification not possible with larger molecules used in recognition-based probe models.

Enzymatically stimulated removal of a fluorescence quencher from a fluorescence probe is a potentially significant and unexplored route for turn-on probes. Such probes may provide selectivity and sensitivity based upon the activities of an endogenous enzyme or cofactor. In particular, this method may employ probes having reporter fluorescence quenched by photoinduced electron transfer (PeT) from a covalently attached enzyme substrate. Fluorescence may ensue from the reporter upon removal of the quencher substrate by an endogenous, cytosolic, disease-associated enzyme found in diseased cells from a wide range of origins.

NAD(P)H:quinone oxidoreductase isozyme I (NQO1) is an enzyme intimately involved with cancer. NQO1 is a gatekeeper for 20S proteasomal degradation of the p53, p73a, and p33 tumor suppressors. NQO1 is also present in a diverse group of human tumor cells (e.g., pancreas, colon, breast, lung, liver, stomach, kidney, head/neck, and ovaries) at levels 2- to 50-fold greater than in normal tissue. Furthermore, NQOI content/activity in tumor cells is strongly affected by cell life cycle and therapeutic approaches. Therefore, NQO1 provides a good target for identifying diseased cells and/or tissues, which will often express increased levels of the enzyme. Importantly, NQOI is found in the cytosol and catalyzes the strict two-electron reduction of quinones to hydroquinones. Moreover, other intracellular or extracellular enzymes and cofactors may be expressed at heightened levels in cancer tissues and/or inflammatory diseases.

Current fluorescent probes for the detection of disease and/or cancer are always-on probes and generally require additional steps, including washing, to determine the presence of disease. The probes generally are not selective and are often expensive, including costs associated with the numerous steps required by the methods utilizing said probes.

Accordingly, there is need for a turn-on probe having a fluorescence reporter that may be activated by intracellular or extracellular enzymes and/or cofactors expressed at heightened levels in cancer tissues and/or inflammatory diseases.

### SUMMARY

In an exemplary embodiment of the present invention, a compound is provided having a formula:

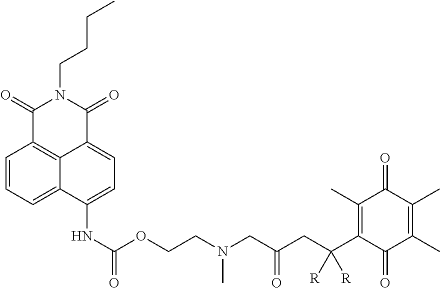

wherein R=Me.

In an exemplary embodiment of the present invention, a compound is provided having one of formulas 1, 2, and 3:

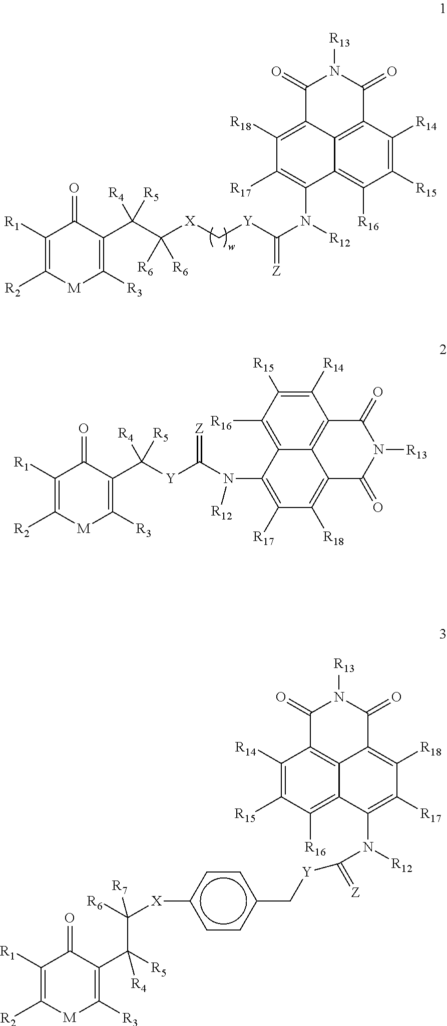

R¹, R², R³, R⁴, R⁵, R⁶, R⁷, R⁸, R¹⁴, R¹⁵, R¹⁶, R¹⁷, R¹⁸ represent, independently, H, Cl, Br, I, CH₃, n-C_{y}H_{2y+1} (where y is an integer value from 1 to 3), n-CⱼH₂ⱼ₊₁O (where j is an integer value from 1 to 3), or (EO)_{z}—R⁹ (where EO is ethylene oxide and z is an integer value from 3 to 100). R⁹ is H, CH₃ (“methyl”), or CF₃CH₂OC(O)CH₂. M is CH₂, —C(O)— (“carbonyl”), or CH—R⁸. X is —C(O)NR¹⁰— (“C-amide”), —C(S)NR¹⁰— (“C-thioamide”), —C(O)O— (“C-ester”), —C(O)S— (“C-thioester”), —C(O)NR¹⁰C(O)— (“imide”), —C(O)OC(O)— (“anhydride”), —CH₂OC(O)— (“O-ester”), —CH₂SC(O)— (“S-thioester”), —CH₂NR¹⁰C(O)— (“N-amide”), —CH₂NR¹⁰C(S)— (“N-thioamide”), —CH₂OC(O)O— (“carbonate”), —CH₂NR¹⁰C(O)NR¹¹— (“urea”), —CH₂NR¹⁰C(S)NR¹¹— (“thiourea”), —CH₂OC(O)— (“O-ester”), —CH₂OC(O)NR¹⁰— (“O-carbamate”), —CH₂NR¹⁰C(O)O— (“N-carbamate”), —CH₂NR¹⁰C(O)S— (“N-thiocarbamate”), —CH₂SC(O)NR¹⁰— (“S-thiocarbamate”), —CH₂OS(O)(O)— (“mesylate”), or —CH₂OP(O)(O)O— (“phosphate”). R¹⁰, R¹¹, R¹², R¹³ represent, independently, H, CH₃, or n-C_{y}H_{2y+1} (where y is an integer value from 1 to 3), or or (EO)_{z}—R⁹ (where EO is ethylene oxide and z is an integer value from 3 to 100); R⁹ is H, CH₃ (“methyl”), or CF₃CH₂OC(O)CH₂. Y and Z represent, O and O (“carbamate”), S and O (“S-thiocarbamate”), N and O (“urea”), or N and S (“thiourea”). W is an integer value of 1 or 2, indicating the number of methylenes (—CH₂—).

In an exemplary embodiment of the present invention, a cloaked fluorophore is provided having: a fluorescent napthalimide reporter; a quinoidal moiety; and a linker. The linker links the quinoidal moiety to the reporter.

In an exemplary embodiment of the present invention, a method of detecting diseased cells is provided, including: administering to cells a compound of formula

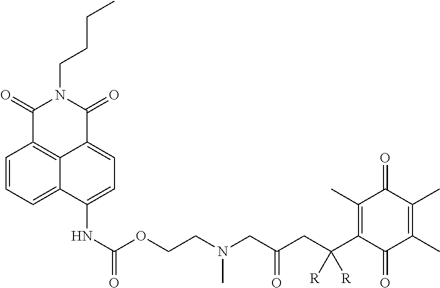

and analyzing the cells for fluorescence. R=Me. Fluorescence indicates disease. The diseased cells may be cancerous. The diseased cells may express NAD(P)H:quinone oxidoreductase isozyme I. The method may include analyzing the cells under a fluorescent microscope. The method may include analyzing the cells with multiphoton microscopy imaging. The diseased cells may be circulating tumor cells. The method may include analyzing the cells with a flow cytometer.

In an exemplary embodiment of the present invention, a method of delineating boundaries of a cancerous tumor in tissue is provided, including: administering to the tissue a compound of formula

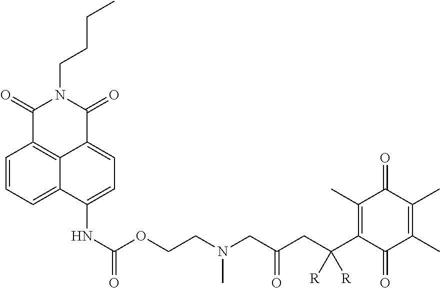

analyzing the tissue for fluorescence; and identifying boundaries between portions of tissue expressing fluorescence and portions of tissue not expressing fluorescence.

### DESCRIPTION OF DRAWINGS

FIG. 1 is formula for a cloaked fluorophore according to an exemplary embodiment of the present invention.

FIG. 2 is a synthetic scheme for the cloaked fluorophore shown in FIG. 1.

FIG. 3 is a schematic representation of PeT quenching in a cloaked fluorophore and emission properties of the cloaked fluorophore according to an exemplary embodiment of the present invention.

FIG. 4 shows three chemical formulas for fluorescent markers according to exemplary embodiments of the present invention.

FIG. 5 shows cyclic voltammograms for tri-methyl locked quinone species according to an exemplary embodiment of the present invention.

FIG. 6 is a graph comparing dithionite-initiated formation of a fluorescent reporter from Q₃NI and Q₁NI according to an exemplary embodiment of the present invention.

FIG. 7 is a kinetics plot of hNQO1 toward a Q₃NI probe according to an exemplary embodiment of the present invention.

FIG. 8 is a graph showing cytometry assay of Q₃NI activation according to an exemplary embodiment of the present invention.

### DETAILED DESCRIPTION

Embodiments of the present disclosure provide cloaked fluorophores which may be activated by intracellular or extracellular enzymes or upregulated cofactors. Embodiments may be activated by enzymes or cofactors having increased activity associated with diseased cells, such as cancer cells. Embodiments may incorporate a naphthalimide-based fluorophore cloaked by a quinone group that may be removed upon enzymatic activation. The fluorophore and quinone group may be bound by a linker, such as ethanolamines or substituted aminobenzyl alcohols. Embodiments of the present invention provide methods of identifying and quantifying cancer-related events and targets for future personalized oncology. Embodiments of the present invention provide synthetically accessible and economically viable probe molecules fully able to report the presence of disease events and targets in a rapid and highly selective and sensitive fashion.

In example embodiments, there is provided a PeT-quenched fluorescence probe which may be activated by NQO1. Embodiments of the fluorescence probe may be hydrophobic and small in molecular weight, allowing the fluorescence probe to readily penetrate membranes of cancer cells. Embodiments may undergo rapid and preferential, two-electron reduction of a quinone quencher subunit, allowing intensely light-emissive reporter to fluoresce. Efficacious PeT quenching of fluorescence prior to subunit self-cleavage may be ensured by selection of electronic properties of a naphthalimide reporter and a quinone quencher. Embodiments of the highly fluorescent reporter may be retained by cells, and may exhibit strong a Stokes shift between absorption and fluorescence emission maxima due to a push-pull internal charge transfer mechanism associated with the naphthalimide scaffold. Novel characteristics of the present invention may allow for rapid and enhanced signal-to-background imaging and detection of living cancer cells without the typical requirement of unactivated probe removal from the environment. Embodiments of the profluorogenic probe may provide real-time, highly sensitive, and selective human tumor cell analysis and differentiation based on NQO1 content.

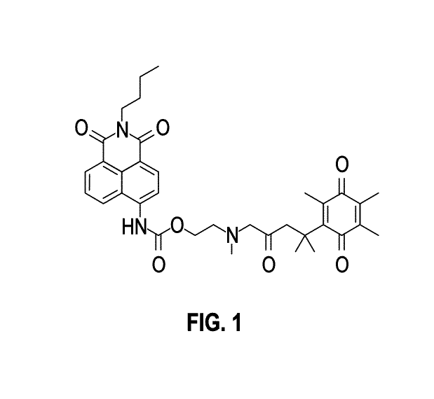

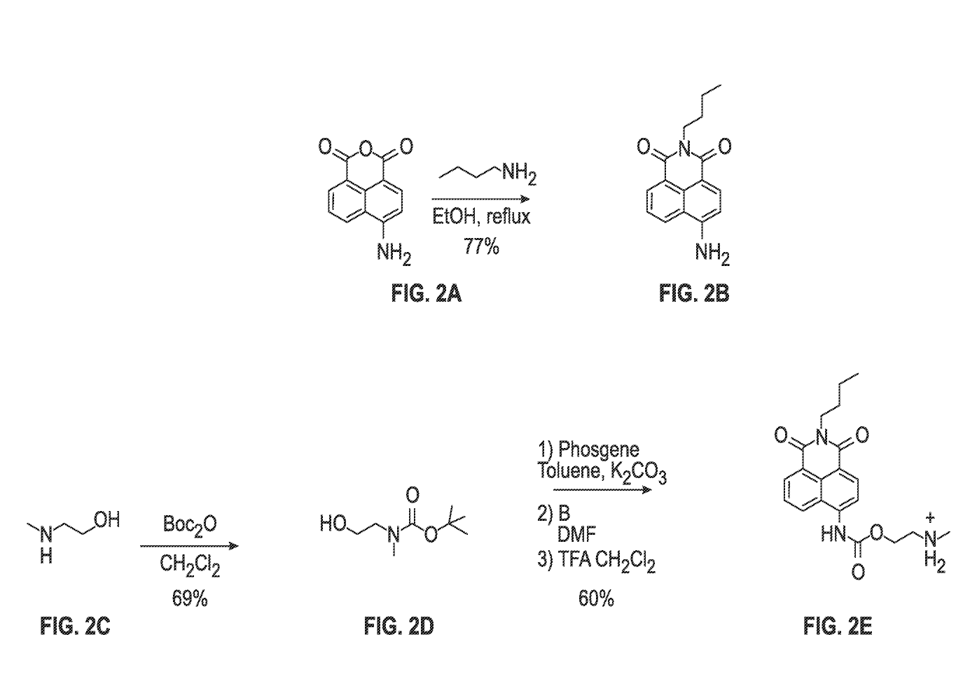

In an example embodiment of the present disclosure, there is provided a novel compound of the formula shown in FIG. 1. The formula may include a fluorescent napthalimide reporter; a quinone moiety; and an ethanolamine linker linking the quinone moiety to the fluorescent napthalimide reporter. Embodiments further include methods of synthesizing the novel compound shown in FIG. 1. FIG. 2 shows a synthetic scheme for synthesizing the novel compound shown in FIG. 1 according to an example embodiment of the present invention.

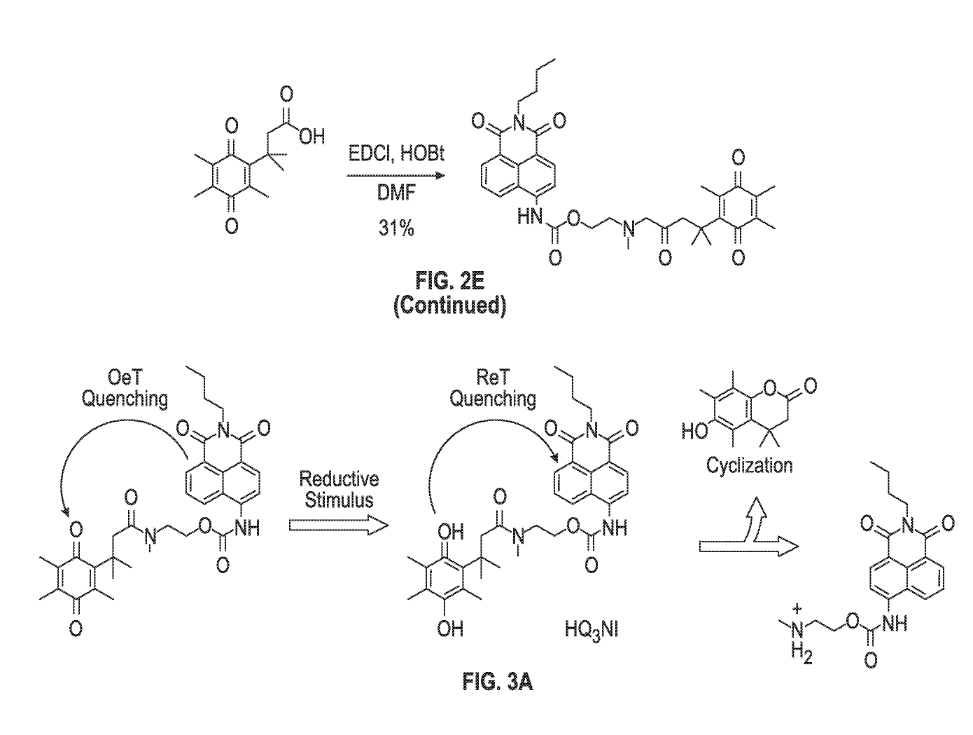

Embodiments of the present invention provide a probe (Q₃NI) having fluorescence signal of a naphthalimide reporter quenched via oxidative electron transfer (OeT) by a covalently attached quinone propionic acid motif. See, e.g. FIG. 3A. The Q₃NI provides the novel mechanism of reporter quenching by carefully tuning the electronic and optical properties of the quinone (Q₃) OeT quencher and the naphthalimide reporter (NI), due to extant naphthalimide reporters that are quenched by reductive electron-transfer (ReT). The Rehm-Weller equation,

$$Δ {G}_{P e T} = {E}_{D} - {E}_{A} - Δ {G}_{00} - \frac{{e}^{2}}{ɛ d}$$

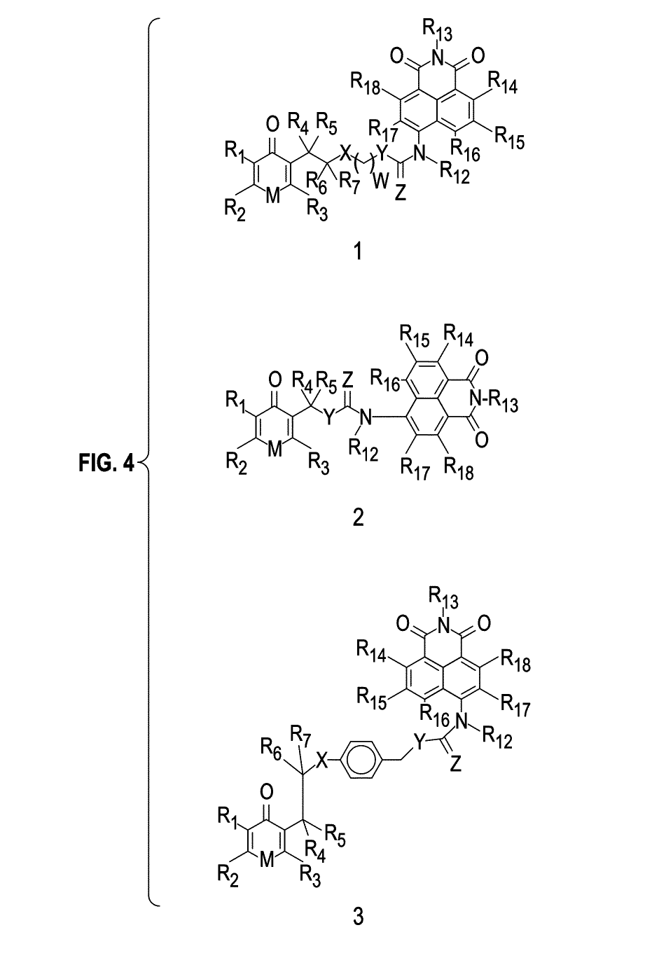

was used to examine possible quinone propionic acid quenchers and 1,8-naphthalimide reporters as well as linkers between the OeT quencher and the NI reporter, so as to ensure that quenching is thermodynamically feasible and efficient. In this equation, E_{D} is the redox potential of the donor and E_{A} that of the acceptor, ΔG₀₀ is the energy of the first excited singlet state of the reporter, and e²/εd is the Coulombic interaction energy of the ion pair, known to be 0.06 eV. The energy of the first excited singlet state of NI was measured to be 3.06 eV. From voltammetric measurements, E_{D} of NI was determined to be 1.74 V, and E_{A} for the quinone propionic acid group of Q₃NI was found to be −1.01 V. See, FIG. 5. From these values, the energy change for this OeT process, ΔG_{PeT}, is calculated to be −0.37 eV, indicating that electron transfer from the excited dye to the electron-poor quinone is thermodynamically favorable. The quinone was attached to the naphthalimide via an N-methylethanolamine linker through a carbamate to the amine of the naphthalimide ring. This linker imparts three properties on Q₃NI: the linker is sufficiently short to allow for a high probability of electron transfer, the electron-withdrawing carbamate yields a favorable ΔG_{PeT}, and the presence of the tertiary amide provides enhanced environmental stability. As a result, the fluorescence of Q₃NI in pH 7.4, 0.1 M PBS is effectively quenched in comparison to that of the free NI reporter, as noted by their spectra in FIGS. 3B and 3C and respective fluorescence quantum yields Φ of 0.007 and 0.23 obtained using quinine sulfate as standard. The quantum yield for NI is superior or comparable to that of other dyes applied to cancer detection and localization, such as Φ=0.0028 for indocyanine green and Φ=0.21 for CyS.S dyes. The 33-fold fluorescence enhancement for NI versus Q₃NI and very large Stokes shift of 116 nm (λ_{max abs}=374 run, λ_{max em}=490 nm) bode well for use of Q₃NI as a multifunctional turn-on probe for sensing and imaging applications that utilize reductive stimuli capable of initiating removal of the reduced quinone group.

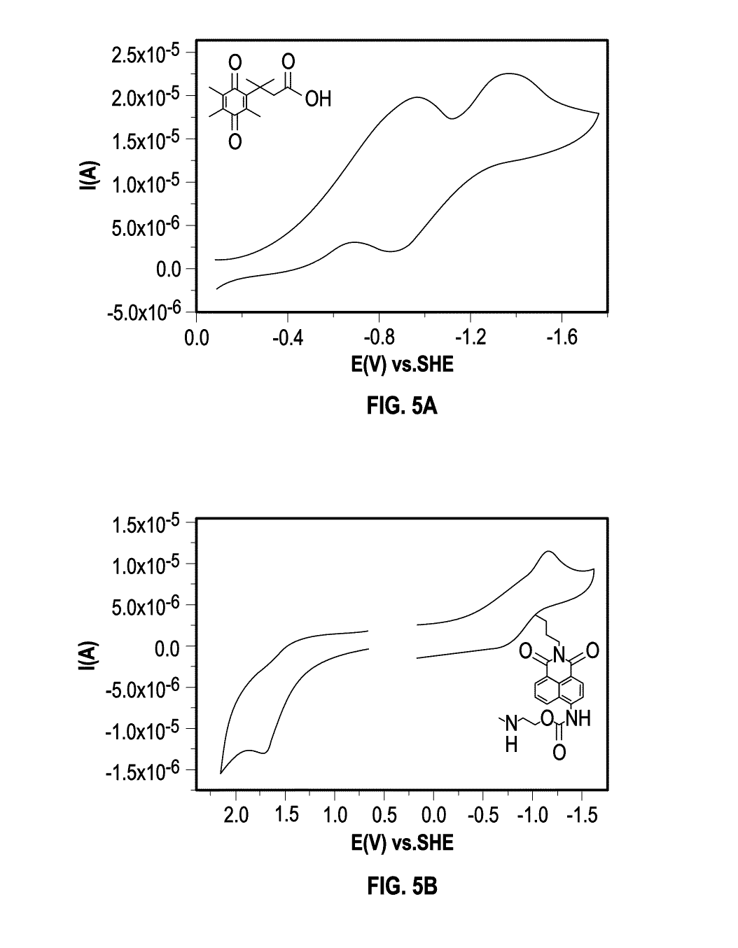

In embodiments of the present invention, fluorescence dequenching of Q₃NI is achieved by reduction-initiated removal of quinone. To determine if it is possible to produce the NI reporter from the Q₃NI probe by the cyclizative cleavage reaction of the hydroquinone via the gem-dialkyl effect that occurs subsequent to two-electron reduction of the quinone, strong reducing agent sodium dithionite was added to aqueous solutions of Q₃NI. Under these conditions, it was found that NI is rapidly released as indicated by the increase in time-dependent fluorescence intensity (FIG. 6) at 470 nm (λₑₓ=370 nm). To confirm that the increase in fluorescence for reduced Q₃NI results from cyclizative cleavage of the hydroquinone reduction product as the lactone, a second probe (Q₁NI) was synthesized such that the rate of lactone formation from its hydroquinone form is >10³ times slower than in the case of reduced Q₃NI, due to the lack of the two methyl groups on the geminal carbon. Dithionite reduction of the Q₁ group is known to be as equally fast as the Q₃ group. As seen in FIG. 6, reduced Q₃NI exhibits exceedingly rapid NI reporter production in comparison to reduced Q₁NI; at 37 min (the maximum recorded for Q₃NI), the signal for reduced Q₃NI is 20-fold higher than that for Q₁NI. This result is supported by a significantly favorable ΔG_{PeT} of −2.87 eV for the reductive quenching process of the NI reporter by the hydroquinone. That is, once the quinone is reduced to its hydroquinone (HQ₃NI, see, FIG. 1A), quenching of reporter fluorescence occurs by ReT, a common feature with naphthalimide dyes. Furthermore, the NI reporter from Q₃NI solutions has been isolated and identified. Thus, the rapid increase in fluorescence for reduced Q₃NI results from naphthalimide dequenching caused by reduction-initiated removal of the quinone propionic acid group by lactonization. Due to the unique quenching mechanism of the Q₃NI turn-on probe sensor, pronounced fluorescence signal enhancement upon revealing the NI reporter and its large Stokes shift, and the known resistance of the quinone propionic acid trigger group to reduction by other biological species, Q₃NI was investigated as a sensor probe of human NAD(P)Rquinone oxidoreductase isozyme 1 (hNQO1) activity in real-time biological applications.

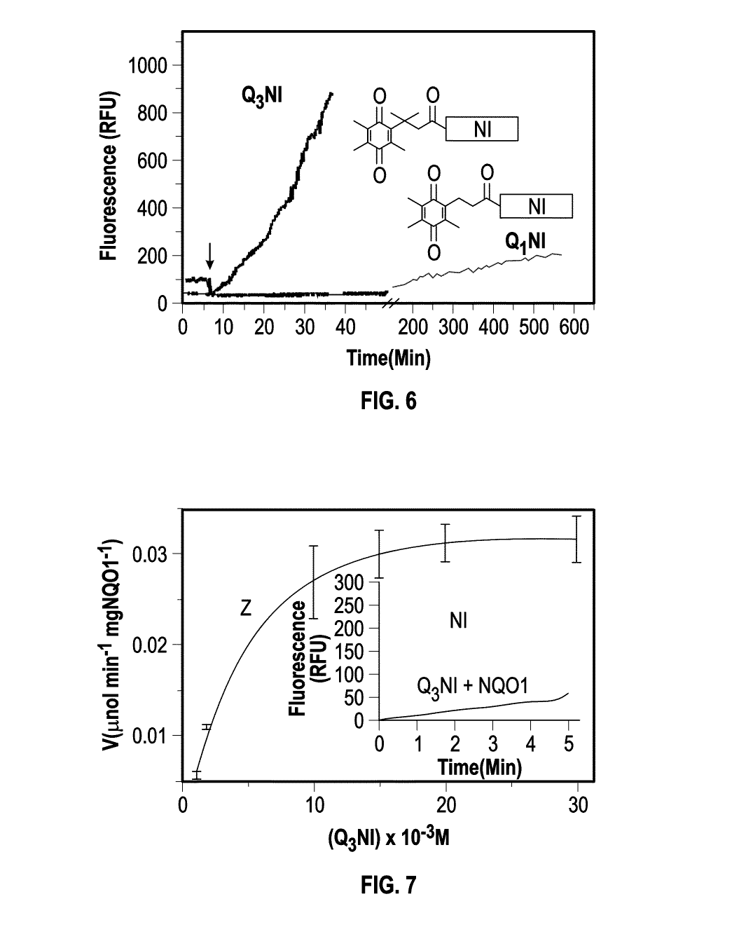

In embodiments of the present in invention hNQO 1 reduces fluorescent probes cloked with a quinone moiety. In embodiments of the present invention, Q₃NI is a probe capable of activation by hNQO1 to yield the NI reporter at a significant rate. Apparent kinetic parameters of Q₃NI were obtained from Michaelis-Menten kinetic treatment of the time-dependent NI reporter production, namely the Michaelis constant (Kₘ), maximum velocity (Vₘₐₓ), catalytic constant (k_{cat}), and substrate specificity (k_{cat}/Kₘ). The high rate of NI reporter production under in vitro conditions is readily apparent in FIG. 7; after 5 min, a fluorescent signal has been attained that is 22% of the maximum achievable, yielding 2.2×10⁻⁷ M of released reporter. From the plot shown in FIG. 7, Kₘ=3.86±0.79 μM, Vₘₐₓ=0.037±0.002 μmol min⁻¹ mg·NQO⁻¹, k_{cat}=0.019±0.001 s⁻¹, and k_{cat}/Kₘ=4.94±0.33×10³ M⁻¹ s⁻¹. Due to the presence of the nonbulky ethanolarnine linker and the need of only a single activation step to reveal reporter fluorescence, the kinetic constants are significantly higher than those of other NQO1 activatable fluorophores, thereby ensuring sufficient signal enhancement for fast detection of hNQO1 activity.

Embodiments of the present invention may allow for cancer cells to be rapidly visualized and differentiated. The colorectal carcinoma cell line HT-29 and the nonsmall cell lung cancer (NSCLC) A549 cell line are known to possess significant hNQO1 activity, while the NSCLC H596 cell line has been reported to have undetectable hNQO1 activity. After a 10 min incubation period in a cell culture solution containing 2×10⁻⁵ M Q₃NI, it was possible to differentiate between the various substrate-cultured cells (4.84 cm²) using only a handheld fluorescent lamp emitting at 365 nm and the unaided eye. Both HT-29 (3.69×10⁶ total cells) and A549 (5.72×10⁶ total cells) appeared fluorescent blue, while H596 (3.96×10⁶ total cells) exhibited no apparent emission. The ability to visually determine the presence of hNQO1 in a small number of cells is due to the marked difference in fluorescence from the unquenched NI reporter (Φ=0.23) and quenched Q₃NI probe (Φ=0.007) and the large Stokes shift of the NI reporter that results in fluorescence emission in the visible spectrum (400 nm to ˜600 nm). These outcomes point to use of the Q₃NI probe sensor in the real-time, visible (without the aid of imaging equipment), and accurate determination of tumor/healthy tissue borders so as to allow for surgical resection of tumors with small foci.

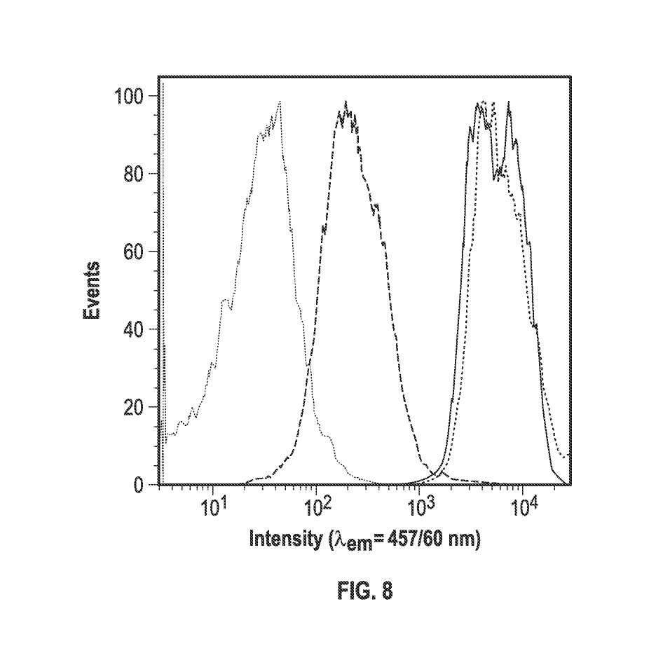

Flow cytometry assays were used to assess the applicability of the Q₃NI probe to rapidly detect and quantify tumor cells containing hNQO1. See, FIG. 8. The probe was incubated in a suspension of HT-29, A549, H596, and H446 (a small-cell lung carcinoma known to be devoid of hNQO1 activity) cells for either 10 min or 60 min, and a flow cytometer was used to measure fluorescence (λₑₓ=405 nm, λₑₘ=457/60 nm) in 10,000 individual cells. FIG. 8 shows the histograms for each cancer cell line and demonstrates that a high-intensity, unimodal distribution of signals is obtained for Q₃NI activation in each of the two hNQO1-positive cell lines (HT-29 and A549), while the negative cell lines H596 and H446 produced minimal fluorescence. It was also found that there was little change in the cell count or intensity of the histograms for a longer probe incubation time (60 min vs 10 min, FIG. 8), demonstrating the rapid and substantial activation of Q₃NI in A549 and HT-29 cells. Importantly, the sustained low fluorescence observed with the H446 cells (FIG. 8) points to the intracellular stability of Q₃NI (lack of nonspecific activation). Thus, it is indicated that Q₃NI is a highly sensitive and selective probe capable of being used to rapidly discern different types of tumor cells in fluidic streams.

In agreement with flow cytometry data, wide-field imaging of fixed hNQ01-positive cells exposed to the Q₃NI probe of the present invention for 10 min revealed significant probe uptake and activation that leads to intracellular NI fluorescence for the A549 and HT-29 cell lines; however, minimal signal was observed in the hNQ01-negative H596 cells. There was no indication of reporter in the nucleus, pointing to the lack of NQOI there; this is in contrast to previous work using immunohistochemical staining of penneabilized, fixed cells. The average cytosolic signal was 9 times higher in A549 cells versus NQOI-negative H596 cells, while it was 23 times higher in HT-29 cells compared to the NQOI-negative H596 cells. After incubating live HT-29 cells for 20 min with Q₃NI followed by exposure to acidic organelle-specific Lysotracker Red in the media in the imaging dish, it was found that the majority of the NI signal originated from the cytosolic region. Accumulation of the basic (secondary amine pKₐ ˜11) NI occurred in acidic late endosomal and lysosomal vesicles, a beneficial outcome that leads to enhanced intracellular retention of NI. The higher signal-to-background value achieved for activation of Q₃NI to NI reporter (9- to 23-fold) in target versus nontarget cells, relative to that of other exogenously introduced sensor probes for whole tumor analysis (2.5- to 5-old), points to the potential of Q₃NI to provide highly selective tumor cell analyses with low limits of detection, even in the face of possible background fluorescence from hemoglobin and other species. In addition, results indicate that dye quantum yield is affected little, and there is no apparent efflux of NI reporter from cells during paraformaldehyde fixing, as noted by sustained fluorescence in fixed samples stored for 10 months in the laboratory ambient. Collectively, there is great potential for use of our probe/reporter system for ex vivo quantitative analysis of excised tumor cells and long-term in vivo and in vitro imaging.

Multiphoton (MP) microscopy imaging of cells and tissues may be more advantageous than traditional fluorescence microscopy, because use of the characteristic long-wavelength photons offers higher fluorophore and cellular photostability that provides extended imaging duration, less background signal from out-of-focus excitation and scattering events, and deeper penetration depth. In addition, MP imaging is ideal for direct observation of targets in their physiological environment and ex vivo thick-specimen sampling where 2D and 3D maps can be generated. During incubation with complete growth medium containing 2.0×10⁻⁵ M Q₃NI, 2-photon microscopy revealed significant fluorescence signal from NI in living, hNQO1-positive HT-29 and A549 cells and minimal signal in two living, hNQO1-negative cell lines, H596 and H446. The average fluorescence signal was determined to be 13-fold higher in A549 cells compared to H596 cells, and 3.66×10⁴-fold higher versus H446 cells. Similar results were obtained with the HT-29 cell line, with the cytosolic intensity being 15- and 4.51×10⁴-fold higher compared to H596 and H446 cells. As before, the signal appears somewhat heterogeneous throughout the cytosolic space of the HT-29 and A549 cells due to NI accumulation in acidic organelles. To ensure Q₃NI and NI had little effect on cell health, cells were incubated in a 2.0×10⁻⁵ M Q₃NI solution in complete growth medium for 1 h and 1 day, and then cell viability was assessed with a trypan blue assay. After 1 h, cell viability for HT-29, A549, and H596 was 97.7%, 98.8%, and 100%, while it was 97.7%, 98.7%, and 98.4% after 24 h. Of particular importance is the real-time nature of the Q₃NI probe in imaging, as this system does not require the time-consuming wash steps characteristic of always-on reporters.

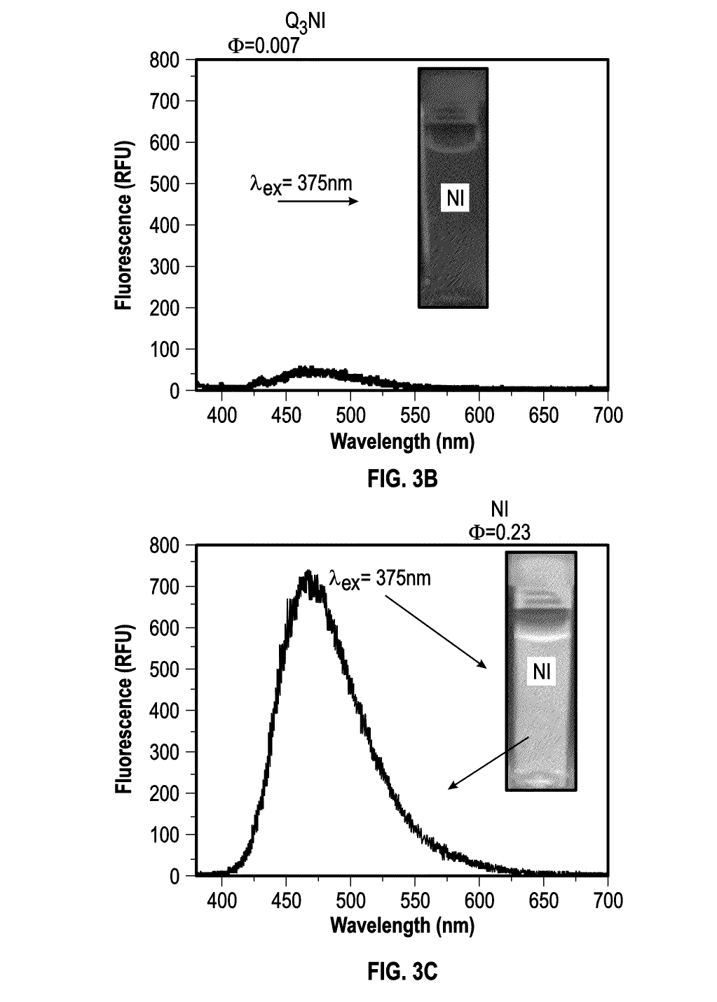

Alternative embodiments of a cloaked fluorescent probe are contemplated by the present invention. Embodiments of the present invention may be a compound represented by Formula 1, 2, or 3 of FIG. 4. As shown, R¹, R², R³, R⁴, R⁵, R¹⁰, and R¹¹ of Formula 1, 2, and/or 3 may be CH₃. R⁶, R⁷, R¹², R¹⁴, R¹⁵, R¹⁶, R¹⁷, R¹⁸ may be H. R¹³ may be n-CH₃(CH₂)₃. M may be —C(O)—. X may be —C(O)NH— and w may be 1 or 2. Y may be O and Z may be O.

Embodiments may be activated to fluoresce by NQO1 which may be present in diseased cells such as cancerous cells. Embodiments may be activated to fluoresce by intra- and/or extracellular enzymes and/or cofactors present in cancerous tissues and/or inflammatory diseases. FIG. 3 provides a schematic representation of PeT quenching in a probe of the present invention and emission properties of the probe, according to an example embodiment of the present invention. As shown, fluorescence of the probe is quenched until the probe is exposed to a reductive stimulus. Embodiments may be activated by NQO1 which may cause the latent probe to fluoresce. Embodiments having an ethanolamine linker may be activated by a single activation step to reveal fluorescence. Embodiments allow for increased efficiency and detection of NQO1 activity or cofactor activity.

Embodiments of the present invention include methods for in vivo detection of cancer cells. Embodiments include methods of detecting circulating tumor cells (CTCs) which may include a probe of the present invention as part of a flow cytometry assay. Embodiments may allow for enumeration of cancer cells. Further embodiments include methods of imaging cancer cells and/or tissues, which methods may be incorporated into various medical and/or surgical procedures or strategies to treat cancer. In still further embodiments, methods are provided to image inflammatory diseases, cells, and/or tissues having elevated NQO1 activities or cofactor activities. Embodiments may be employed to localize and/or image various diseases and/or diseased tissues. In still further embodiments, methods are provided for early detection of cancer and/or increased NQO1 activities.

While the embodiments are described with reference to various implementations and exploitations, it will be understood that these embodiments are illustrative and that the scope of the inventions is not limited to them. Many variations, modifications, additions, and improvements are possible. Further still, any steps described herein may be carried out in any desired order, and any desired steps may be added or deleted. Support for the present invention, including example embodiments of the present invention, may be found in the attached documents and figures, all of which arc expressly incorporated herein in their entirety by reference hereto.

What is claimed is:

1. A compound of formula: 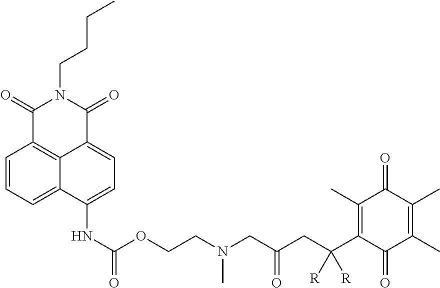
wherein R=Me.

2. A compound, comprising one of formulas 1, 2, and 3: 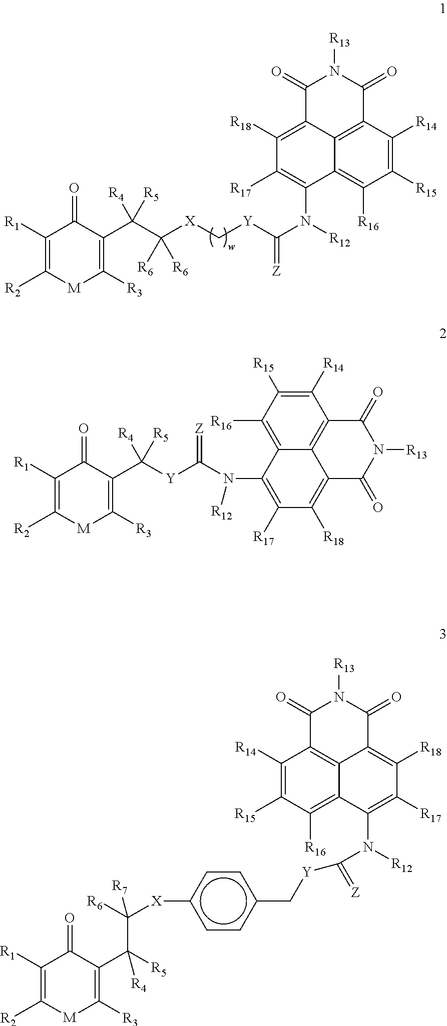
1. wherein, R¹, R², R³, R⁴, R⁵, R⁶, R⁷, R⁸, R¹⁴, R¹⁵, R¹⁶, R¹⁷, R¹⁸ represent, independently, H, Cl, Br, I, CH₃, n-C_{y}H_{2y+1} (where y is an integer value from 1 to 3), n-CⱼH₂ⱼ₊₁O (where j is an integer value from 1 to 3), or (EO)_{z}—R⁹ (where EO is ethylene oxide and z is an integer value from 3 to 100);
wherein, R⁹ is H, CH₃ (“methyl”), or CF₃CH₂OC(O)CH₂;
wherein, M is CH₂, —C(O)— (“carbonyl”), or CH—R⁸;
wherein, X is —C(O)NR¹⁰— (“C-amide”), —C(S)NR¹⁰— (“C-thioamide”), —C(O)O— (“C-ester”), —C(O)S— (“C-thioester”), —C(O)NR¹⁰C(O)— (“imide”), —C(O)OC(O)— (“anhydride”), —CH₂OC(O)— (“O-ester”), —CH₂SC(O)— (“S-thioester”), —CH₂NR¹⁰C(O)— (“N-amide”), —CH₂NR¹⁰C(S)— (“N-thioamide”), —CH₂OC(O)O— (“carbonate”), —CH₂NR¹⁰C(O)NR¹¹— (“urea”), —CH₂NR¹⁰C(S)NR¹¹— (“thiourea”), —CH₂OC(O)— (“O-ester”), —CH₂OC(O)NR¹⁰— (“O-carbamate”), —CH₂NR¹⁰C(O)O— (“N-carbamate”), —CH₂NR¹⁰C(O)S— (“N-thiocarbamate”), —CH₂SC(O)NR¹⁰— (“S-thiocarbamate”), —CH₂OS(O)(O)— (“mesylate”), or —CH₂OP(O)(O)O— (“phosphate”);
wherein, R¹⁰, R¹¹, R¹², R¹³ represent, independently, H, CH₃, or n-C_{y}H_{2y+1} (where y is an integer value from 1 to 3), or or (EO)_{z}—R⁹ (where EO is ethylene oxide and z is an integer value from 3 to 100); R⁹ is H, CH₃ (“methyl”), or CF₃CH₂OC(O)CH₂;
wherein, Y and Z represent, O and O (“carbamate”), S and O (“S-thiocarbamate”), N and O (“urea”), or N and S (“thiourea”); and
wherein w is an integer value of 1 or 2, indicating the number of methylenes (—CH₂—).

3. A cloaked fluorophore, comprising:
a fluorescent napthalimide reporter;
a quinoidal moiety; and
a linker;
wherein the linker links the quinoidal moiety to the reporter.

4. A method of detecting diseased cells, comprising:
administering to cells a compound of formula 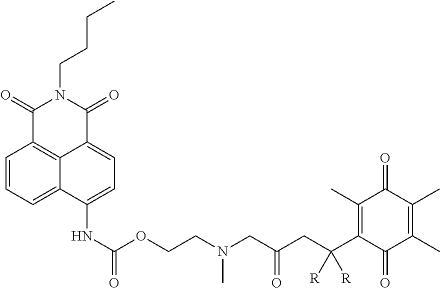
analyzing the cells for fluorescence;
wherein R=Me; and
wherein fluorescence indicates disease.

5. The method of claim 4, wherein the diseased cells are cancerous.

6. The method of claim 4, wherein the diseased cells express NAD(P)H:quinone oxidoreductase isozyme I.

7. The method of claim 4, further comprising:
analyzing the cells under a fluorescent microscope.

8. The method of claim 4, further comprising:
analyzing the cells with multiphoton microscopy imaging.

9. The method of claim 4, where the diseased cells are circulating tumor cells.

10. The method of claim 9, further comprising:
analyzing the cells with a flow cytometer.

11. A method of delineating boundaries of a cancerous tumor in tissue, comprising:
administering to the tissue a compound of formula 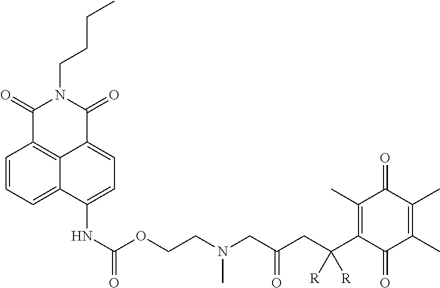
Analyzing the tissue for fluorescence; and
identifying boundaries between portions of tissue expressing fluorescence and portions of tissue not expressing fluorescence.
